(19) United States
(12) Patent Application Publication
(10) Pub. No.: US 2022/0259114 A1
(43) Pub. Date: Aug. 18, 2022

(54) Microbe-Based Compositions for Restoring Soil Health and Controlling Pests

(71) Applicant: Locus Agriculture IP Company, LLC, Solon, OH (US)

(72) Inventors: Sean FARMER, Ft. Lauderdale, FL (US); Ken ALIBEK, Solon, OH (US)

(21) Appl. No.: 17/625,835

(22) PCT Filed: Aug. 12, 2020
(86) PCT No.: PCT/US2020/045845

§ 371 Date: Jan. 10, 2022

Related U.S. Application Data
(60) Provisional application No. 62/885,455, filed on Aug. 12, 2019.
(60) Provisional application No. 62/953,632, filed on Dec. 26, 2019.

(51) Int. Cl. C05F 11/08; A01N 63/22; A01N 63/32; A01N 63/20; A01P 15/00; A01N 63/50; C05F 11/02; C05G 3/50

(52) U.S. Cl. CPC C05F 11/08; A01N 63/22; A01N 63/32; C05G 3/50; A01P 15/00; A01N 63/50; C05F 11/02; A01N 63/20

(57) ABSTRACT

Compositions and methods are provided for enhancing soil health and/or plant health. In particular, the subject invention relates to compositions comprising microbes and/or their growth by-products for use in improving fertility, salinity, water retention, and other soil characteristics, as well as controlling pests and stimulating the growth of plants. In certain embodiments, the growth by-products are biosurfactants.

### CROSS-REFERENCE TO RELATED APPLICATION

This application claims priority to U.S. Provisional Patent Application Ser. Nos. 62/885,455, filed Aug. 12, 2019, and 62/953,632, filed Dec. 26, 2019, both of which are incorporated herein by reference in their entirety.

### BACKGROUND OF THE INVENTION

In the agriculture industry, certain common issues hinder the ability of farmers to maximize production while keeping costs low. These include, but are not limited to, infections and infestations caused by bacteria, fungi, and other pests and pathogens; the high costs of chemical fertilizers and herbicides, including their environmental and health impacts; and the difficulty for plants to efficiently absorb nutrients and water from different types of soil.

Efficient nutrient and water absorption in particular are crucial for producing crops that thrive, especially in different geographic areas with soil types that have certain unsuitable qualities for growing crops. There are several different types of soil, determined by the amount of clay, silt or sand particles present therein.

Clay soil contains a high percentage of clay and silt. The particles are small and cling together, retaining water and nutrients well; however, clay soil is susceptible to compaction, where mineral grains are squeezed together by the weight of the overlying sediment, thus reducing the soil's porosity. This can hinder a plant's roots from penetrating the soil, as well as the ability of moisture and nutrients to reach the roots. Furthermore, clay soil drains slower than other soil types, and in areas that experience cold and freezing temperatures, can take longer to warm or thaw in the spring. Clay soil can be identified by its sticky, slippery consistency, and its tendency to cling to garden tools.

Sandy soil is comprised of larger, coarser particles than clay soil. It has a low capacity for moisture and nutrient retention, so fertilization and watering must occur more frequently than with other types of soils. Sandy soil is typically less fertile than other soil types because there are large gaps between the particles. These gaps allow water and nutrients to drain away more easily. This type of soil can be identified by its rough texture, and its tendency to fall apart rather than stick together when held.

Loam soil has a balance of clay, silt, sand and organic material; making it the most ideal type of soil for gardening purposes, and the most fertile soil for agricultural purposes. It is capable of retaining moisture and nutrients well. Loam is also aerated, meaning air can circulate through the soil and water can drain more easily. It can be identified by its ability to hold its shape when squeezed lightly, and is easier to dig through than other soil types.

Two other soil types, silty soils and peaty soils, are known for creating difficulties with water drainage. Silty soils usually have a moisture retention capacity similar to loam; however, depending on the clay-to-silt ratio, water may drain more slowly. Peaty soil is most commonly found in marshy, wet climate areas. Though peaty soil is full of nutrients, it is easily susceptible to waterlogging.

The type and composition of soil are important factors in whether or not a particular plant and/or crop will thrive. Sometimes, additives called soil amendments are needed to improve the soil for a particular type of crop based on its specific needs. A soil amendment is a composition that improves the physical and/or chemical characteristics of the soil to which it is applied. Soil amendments can reduce compaction, aerate soil, and allow water and nutrients to move more easily through soil to reach plant roots. Some soil amendments also add nutrients to the soil, modulate salinity and/or help retain moisture.

Soil amendments can comprise organic matter such as sphagnum peat moss, humus, manure, compost, topsoil, and various minerals and sands. Currently, in adding materials to a soil, a balance of materials must be struck so as to provide the soil with proper amounts of water retention and to provide desired amounts of aeration within the soil so as to better enable plant growth. For example, soils must have an adequate amount of water-retaining material, yet at the same time be sufficiently drainable so as to prevent excess water from damaging and hindering plant growth.

Soils must also be sufficiently dense to maintain root structure and support the plant, yet at the same time be sufficiently loosely packed so as to allow roots to expand and support plant growth. In addition, soils must contain appropriate quantities of salts, as well as minerals such as nitrogen, phosphates, calcium, copper, and iron.

In addition to the properties of soil, control of pests is also an important aspect of producing crops. The use of pesticides, however, risks the contamination and pollution of soil and agricultural products, but can be harmful to humans and may unintentionally harm beneficial species. Furthermore, the over-dependence and long-term use of certain chemical pesticides can alter soil ecosystems, reduce stress tolerance, increase pest resistance, and impede plant and animal growth and vitality.

Mounting regulatory mandates that govern the availability and use of chemicals and/or antibiotics, as well as consumer demands for residue free, sustainably-grown food produced with minimal harm to the environment, are impacting the pest-control industry and causing an evolution of thought regarding how to address the myriad of challenges. The demand for safer pesticides and alternate pest control strategies is increasing. While wholesale elimination of chemicals is not feasible at this time, farmers are increasingly embracing the use of biological measures as viable components of Integrated Nutrient Management and Integrated Pest Management programs.

To address the global needs for sustainable methods of producing food and consumable products, microbes such as bacteria, yeast and fungi, as well as their byproducts, are becoming increasingly useful replacements for chemical agricultural applications. For example, farmers are embracing the use of biological agents such as live microbes, bio-products derived from these microbes, and combinations thereof, as soil amendments, biopesticides and biofertilizers. These biological agents are less harmful compared to conventional chemicals, they are more efficient and specific, and they often biodegrade quickly, leading to less soil and environmental pollution.

The economic costs and the adverse health and environmental impacts of current methods of crop production continue to burden the sustainability and efforts of producing food and other crop-based consumer products. Environmental awareness and consumer demand has promoted the search for improved products to, for example, enhance soil characteristics and/or control pests.

### BRIEF SUMMARY OF THE INVENTION

The subject invention provides multi-functional agricultural compositions and methods of their use for enhancing the health of soil, as well as the health of plants growing in the soil. Advantageously, the microbe-based products and methods of the subject invention are environmentally-friendly, non-toxic and cost-effective.

In preferred embodiments, the subject invention provides compositions for enhancing the fertility and/or health of soil. In some embodiments, the compositions can also serve as, e.g., pesticides, plant immune modulators, and/or plant growth stimulants.

In certain embodiments the compositions comprise one or more beneficial microorganisms and/or one or more microbial growth by-products, such as biosurfactants, enzymes and/or other metabolites. The composition may also comprise the fermentation medium in which the microorganism(s) were produced.

The composition can be formulated for applying to soil and/or to above- and below-ground plant parts. For example, in certain embodiments, the composition can be mixed with water and applied to plants and/or to soil via an irrigation system.

The microorganisms may be live and/or inactivated. In preferred embodiments, the beneficial microorganisms are yeasts and/or bacteria. In a specific embodiment, the composition can comprise Starmerella bombicola yeasts.

In some embodiments, yeast extract and/or other microbial hydrolysates produced by methods known in the microbiological arts are included in the composition.

In certain embodiments, the microbial growth by-products are biosurfactants selected from, for example, glycolipids (e.g., sophorolipids, cellobiose lipids, rhamnolipids, mannosylerythritol lipids and trehalose lipids), lipopeptides (e.g., surfactin, iturin, fengycin, arthrofactin and lichenysin), flavolipids, fatty acid esters, phospholipids (e.g., cardiolipins), and high molecular weight polymers such as lipoproteins, lipopolysaccharide-protein complexes, and polysaccharide-protein-fatty acid complexes.

The composition can comprise one or more biosurfactants at a concentration of, for example, 0.001% to 10%, 0.01% to 5%, 0.05% to 2%, and/or from 0.1% to 1% by weight. In certain specific embodiments, the biosurfactants are glycolipids and/or lipopeptides.

The microbe-based compositions of the subject invention can be obtained through cultivation processes ranging from small to large scale. These cultivation processes include, but are not limited to, submerged cultivation/fermentation, solid state fermentation (SSF), and combinations thereof.

In preferred embodiments, the subject invention provides methods for enhancing the health of soil and/or plants, wherein a composition comprising one or more microorganisms and/or one or more microbial growth by-products, such as biosurfactants, enzymes and/or other metabolites, is contacted with the soil and/or the plant. The composition may also comprise the fermentation medium in which the microorganism(s) were produced, such as a submerged fermentation broth or a solid-state substrate.

In certain embodiments, the growth by-products are biosurfactants, such as glycolipids and/or lipopeptides.

The microbes can be either live, dormant or inactive at the time of application. In some embodiments, the microbes are in the form of yeast extract and/or another microbial hydrolysate.

The microbial growth by-products can be those produced by the microorganism(s), and/or they can be applied in addition to the growth by-products produced by the microorganism(s) of the composition.

The methods can further comprise adding materials to enhance microbe growth before, during, and/or after application (e.g., adding nutrients and/or prebiotics). Thus, live microorganisms can grow in situ and produce the active compounds onsite. Consequently, a high concentration of microorganisms and their growth by-products can be achieved easily and continuously in soil.

In some embodiments, the method comprises applying one or more microbial growth by-products to the soil and/or the plant without a microorganism. Specifically, in one embodiment, the method comprises applying a composition comprising purified glycolipid and/or lipopeptide biosurfactants to the soil and/or plant.

In some embodiments, the methods are used for restoring soil health, wherein the soil being treated was once healthy, but deteriorated over some period of time. The restoration may bring the soil back to its previous state of health and/or an enhanced state of health.

In certain embodiments, enhancing soil health comprises one or more of, for example, removing pollutants from the soil, improving the nutrient content and availability of the soil, improving drainage and/or moisture retention properties of the soil, improving the salinity of the soil, improving the diversity of the soil microbiome, and/or controlling a soil-borne pest.

In some embodiments, the methods are used for controlling above-ground and below-ground pests. In some embodiments, the method can be useful for controlling pests such as arthropods, nematodes, protozoa, bacteria, fungi, and/or viruses.

In some embodiments, the methods are used for stimulating the growth of plants and/or improving the plants' ability to outcompete weeds and other detrimental plants.

The microbe-based products can be used either alone or in combination with other compounds for efficiently enhancing soil and/or plant health. For example, in some embodiments, the method comprises applying additional components, such as herbicides, fertilizers, pesticides and/or other soil amendments, to the soil and/or plants. The exact materials and the quantities thereof can be determined by, for example, a grower or soil scientist having the benefit of the subject disclosure.

In certain embodiments, the compositions of the subject invention have advantages over, for example, biosurfactants alone, when the use of entire microbial culture is employed. These advantages can include one or more of the following: high concentrations of mannoprotein as a part of a yeast cell wall's outer surface; the presence of beta-glucan in yeast cell walls; the inclusion of the fermentation broth and/or solid substrate in the composition; and the presence of, for example, proteins, enzymes, nutrients, other metabolites in the composition.

Advantageously, the present invention can be used without releasing large quantities of polluting compounds into the environment. In fact, in some embodiments, the present invention can be used for reducing the emission of greenhouse gases and other atmospheric pollutants through improved agricultural practices. Additionally, the compositions and methods utilize components that are biodegradable and toxicologically safe. Thus, the present invention can be used as a “green” agricultural product.

### DETAILED DESCRIPTION OF THE INVENTION

The subject invention provides microbes, by-products of their growth, such as biosurfactants, as well as methods of using these microbes and their by-products. More specifically, the subject invention provides microbe-based compositions and methods of their use for enhancing the health of soil and/or plants. Advantageously, the microbe-based products and methods of the subject invention are environmentally-friendly, non-toxic and cost-effective.

### Selected Definitions

The subject invention utilizes “microbe-based compositions,” meaning a composition that comprises components that were produced as the result of the growth of microorganisms or other cell cultures. Thus, the microbe-based composition may comprise the microbes themselves and/or by-products of microbial growth. The microbes may be in a vegetative state, in spore or conidia form, in hyphae form, in any other form of propagule, or a mixture of these. The microbes may be planktonic or in a biofilm form, or a mixture of both. The by-products of growth may be, for example, metabolites, cell membrane components, expressed proteins, and/or other cellular components. The microbes may be intact or lysed. In some embodiments, the microbes are present, with growth medium in which they were grown, in the microbe-based composition. The microbes may be present at, for example, a concentration of at least 1×10⁴, 1×10⁵, 1×10⁶, 1×10⁷, 1×10⁸, 1×10⁹, 1×10¹⁰, 1×10¹¹, 1×10¹² or 1×10¹³ or more CFU per gram or per ml of the composition.

The subject invention further provides “microbe-based products,” which are products that are to be applied in practice to achieve a desired result. The microbe-based product can be simply a microbe-based composition harvested from a microbe cultivation process. Alternatively, the microbe-based product may comprise further ingredients that have been added. These additional ingredients can include, for example, stabilizers, buffers, appropriate carriers, e.g., water, salt solutions, added nutrients to support further microbial growth, non-nutrient growth enhancers and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

As used herein, a “biofilm” is a complex aggregate of microorganisms, wherein the cells adhere to each other and/or to surfaces. In some embodiments, the cells secrete a polysaccharide barrier that surrounds the entire aggregate. The cells in biofilms are physiologically distinct from planktonic cells of the same organism, which are single cells that can float or swim in liquid medium.

As used herein, an “isolated” or “purified” compound, e.g., a polynucleotide or polypeptide, is substantially free of other compounds, such as cellular material, genes, gene sequences, amino acids, or amino acid sequences, with which it is associated in nature and/or in which it was produced. “Isolated” in the context of a microbial strain means that the strain is removed from the environment in which it exists in nature and/or in which it is cultivated. Thus, the isolated strain may exist as, for example, a biologically pure culture, or as spores (or other forms of the strain).

As used herein, a “biologically pure culture” is a culture that has been isolated from materials with which it is associated in nature and/or in which it is cultivated. In a preferred embodiment, the culture has been isolated from all other living cells. In further preferred embodiments, the biologically pure culture has advantageous characteristics compared to a culture of the same microbe as it exists in nature. The advantageous characteristics can be, for example, enhanced production of one or more growth by-products.

In certain embodiments, purified compounds are at least 60% by weight the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

A “metabolite” refers to any substance produced by metabolism (e.g., a growth by-product) or a substance necessary for taking part in a particular metabolic process. Examples of metabolites include, but are not limited to, biosurfactants, biopolymers, enzymes, acids, solvents, alcohols, proteins, vitamins, minerals, microelements, and amino acids.

As used herein, “modulate” means to cause an alteration (e.g., increase or decrease).

Ranges provided herein are understood to be shorthand for all of the values within the range. For example, a range of 1 to 20 is understood to include any number, combination of numbers, or sub-range from the group consisting of 1, 2, 3, 4, 5, 6, 7, 8, 9, 10, 11, 12, 13, 14, 15, 16, 17, 18, 19, 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, “reduce” refers to a negative alteration, and “increase” refers to a positive alteration, each of at least 1%, 5%, 10%, 25%, 50%, 75%, or 100%.

As used herein, “reference” refers to a standard or control condition.

As used herein, “surfactant” refers to a compound that lowers the surface tension (or interfacial tension) between two liquids, between a liquid and a gas, or between a liquid and a solid. Surfactants act as, e.g., detergents, wetting agents, emulsifiers, foaming agents, and dispersants. A “biosurfactant” is a surfactant produced by a living organism.

As used herein, “agriculture” means the cultivation and breeding of plants, algae and/or fungi for food, fiber, biofuel, medicines, cosmetics, supplements, ornamental purposes and other uses. According to the subject invention, agriculture can also include horticulture, landscaping, gardening, plant conservation, orcharding and arboriculture. Further included in agriculture is the care, monitoring and maintenance of soil.

As used herein “preventing” or “prevention” of a situation or occurrence means delaying, inhibiting, suppressing, forestalling, and/or minimizing the onset, extensiveness or progression of the situation or occurrence. Prevention can include, but does not require, indefinite, absolute, or complete prevention, meaning the situation or occurrence may still develop at a later time. Prevention can include reducing the severity of the onset of such a situation or occurrence, and/or inhibiting the progression thereof to one that is more severe or extensive.

As used herein, the term “control” used in reference to a pest means killing, disabling, immobilizing, or reducing population numbers of a pest, or otherwise rendering the pest substantially incapable of causing harm and/or reproducing.

As used herein, a “pest” is any organism, other than a human, that is destructive, deleterious and/or detrimental to humans or human concerns (e.g., agriculture, horticulture). In some, but not all instances, a pest may be a pathogenic organism. Pests may cause or be a vector for infections, infestations and/or disease, or they may simply feed on or cause other physical harm to living tissue. Pests may be single- or multi-cellular organisms, including but not limited to, viruses, fungi, bacteria, parasites, arthropods, protozoa and/or nematodes.

As used herein, a “soil amendment” or a “soil conditioner” is any compound, material, or combination of compounds or materials that are added into soil to enhance the physical and/or chemical properties of the soil. Soil amendments can include organic and inorganic matter, and can further include, for example, microorganisms, fertilizers, pesticides and/or herbicides. Nutrient-rich, well-draining soil is essential for the growth and health of plants, and thus, soil amendments can be used for enhancing the growth and health of plants by, e.g., altering the nutrient and moisture content of soil. Soil amendments can also be used for enhancing soil health and/or fertility.

As used herein, “enhancing” means improving or increasing. For example, enhanced soil health means improved physical structure (e.g., porosity, permeability, bulk), improved fertility (e.g., mineral content, nutrient content, organic matter content), improved wettability and/or drainage, improved salinity, improved soil biodiversity, and/or removal or reduction in pollutants and/or pests. In some embodiments, enhanced soil health is dependent upon the characteristics required for a particular crop that is to be grown in the soil. For example, some plants prefer higher drainage, while others prefer wetter soils.

As another example, enhanced plant health means improving the plant's ability grow and thrive, which includes increased seed germination and/or emergence, improved ability to ward off pests and/or diseases, improved ability to survive environmental stressors, such as droughts and/or overwatering, improved ability to reach a desired size and/or mass, increased amounts and/or size of fruits, leaves, roots, extracts and/or tubers per plant, and/or improved quality of fruits, leaves, roots, extracts and/or tubers (e.g., improving taste, texture, brix, chlorophyll content and/or color).

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All references cited herein are hereby incorporated by reference in their entirety.

### Compositions

In preferred embodiments, the subject invention provides compositions for enhancing the health of soil and/or the health of plants growing therein. In some embodiments, the compositions can also serve as, e.g., pesticides, plant immune modulators and/or plant growth stimulators.

In certain embodiments the compositions comprise one or more beneficial microorganisms and/or one or more microbial growth by-products, such as biosurfactants, enzymes and/or other metabolites. The composition may also comprise the fermentation medium in which the microorganism(s) were produced.

The microorganisms can be, for example, bacteria, yeast and/or fungi. These microorganisms may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In one embodiment, the microorganism is a yeast or fungus. Yeast and fungus species suitable for use according to the current invention, include Aureobasidium (e.g., A. pullulans), Blakeslea, Candida (e.g., C. apicola, C. bombicola, C. nodaensis), Cryptococcus, Debaryomyces (e.g., D. hansenii), Entomophthora, Hanseniaspora, (e.g., H. uvarum), Hansenula, Issatchenkia, Kluyveromyces (e.g., K. phaffii), Mortierella, Mycorrhiza, Meyerozyma guilliermondii, Penicillium, Phycomyces, Pichia (e.g., P. anomala, P. guilliermondii, P. occidentalis, P. kudriavzevii), Pleurotus spp. (e.g., P. ostreatus), Pseudozyma (e.g., P. aphidis), Saccharomyces (e.g., S. boulardii sequela, S. cerevisiae, S. torula), Starmerella (e.g., S. bombicola), Torulopsis, Trichoderma (e.g., T. reesei, T. harzianum, T. hamatum, T. viride), Ustilago (e.g., U. maydis), Wickerhamomyces (e.g., W. anomalus), Williopsis (e.g., W. mrakii), Zygosaccharomyces (e.g., Z. bailii), and others.

In certain embodiments, the microorganisms are bacteria, including Gram-positive and Gram-negative bacteria. The bacteria may be, for example Agrobacterium (e.g., A. radiobacter), Azotobacter (A. vinelandii, A. chroococcum), Azospirillum (e.g., A. brasiliensis), Bacillus (e.g., B. amyloliquefaciens, B. circulans, B. firmus, B. laterosporus, B. licheniformis, B. megaterium, B. mojavensis, B. mucilaginosus, B. subtilis), Burkholderia (e.g., B. thailandensis), Frateuria (e.g., F. aurantia), Microbacterium (e.g., M laevaniformans), myxobacteria (e.g., Myxococcus xanthus, Stignatella aurantiaca, Sorangium cellulosum, Minicystis rosea), Paenibacillus polymyxa, Pantoea (e.g., P. agglomerans), Pseudomonas (e.g., P. aeruginosa, P. chlororaphis subsp. aureofaciens (Kluyver), P. putida), Rhizobium spp., Rhodospirillum (e.g., R. rubrum), Sphingomonas (e.g., S. paucimobilis), and/or Thiobacillus thiooxidans (Acidothiobacillus thiooxidans).

In certain embodiments, the composition comprises Starmerella bombicola, which is an effective producer of glycolipid biosurfactants, such as sophorolipids.

In certain embodiments, the composition comprises Saccharomyces cerevisiae, which can be influenced to produce glycolipid biosurfactants, such as sophorolipids and/or rhamnolipids.

In certain embodiments, the composition comprises a lipopeptide-producing bacterium, such as Bacillus mojavensis, which is capable of producing bioemulsifying compounds, proteases, as well as fengycin and/or surfactin biosurfactants.

In certain embodiments, the composition comprises Myxococcus xanthus, a lipopeptide-producing soil bacterium.

In certain embodiments, the composition comprises B. amyloliquefaciens NRRL B-67928, which is capable of producing surfactin, iturin, lichenysin, and fengycin, in addition to organic acids that help solubilize nutrients in soil.

In certain embodiments, the composition comprises Burkholderia thailandensis, which can be influenced to produce rhamnolipid biosurfactants.

Other microbial strains can be used in accordance with the subject invention, including, for example, any other strains having high concentrations of mannoprotein and/or beta-glucan in their cell walls and/or that are capable of producing biosurfactants, enzymes, nutrients, and other metabolites useful for enhancing soil health, controlling pests, and/or enhancing plant health.

The microbe-based compositions of the subject invention can be obtained through cultivation processes ranging from small to large scale. These cultivation processes include, but are not limited to, submerged cultivation/fermentation, solid state fermentation (SSF), and combinations thereof.

In certain embodiments, the microbe-based composition can comprise fermentation broth and/or solid-state substrate containing a microbial culture and/or the microbial metabolites produced by the microorganism and/or any residual nutrients. The product of fermentation may be used directly without extraction or purification of metabolites. If desired, extraction and purification can be easily achieved using standard extraction and/or purification methods or techniques described in the literature.

The composition may be at least, by weight, 1%, 5%, 10%, 25%, 50%, 75%, or 100% broth and/or solid substrate. The amount of biomass in the composition, by weight, may be anywhere from 0% to 100% inclusive of all percentages therebetween, for example, from 5 g/l to 180 g/l or more, or from 10 g/l to 150 g/l.

The microorganisms may be live and/or inactivated. In some embodiments, the composition comprises inactivated microorganisms, for example, in the form of yeast extract and/or another microbial hydrolysate. According to the subject invention, a “hydrolysate” of a microorganism comprises disrupted cell walls/membranes of a deactivated microorganism, along with the cell contents released therefrom. The process of deactivating, or hydrolysis, often causes the release of compounds from the cells and cell walls/membranes, such as metabolites, enzymes, proteins, peptides, free amino acids, vitamins, minerals and trace elements.

Preferably, the mode of inactivating the microorganism does not also inactivate or denature the biochemical(s) it produced during cultivation. Inactivation can be achieved using, for example, boiling, dry-heat oven, autoclaving, pasteurization, refrigeration, freezing, high-pressure processing, hyperbaric oxygen therapy, desiccation, lyophilization, radiation, sonication, HEPA (high-efficiency particulate air) filtration, or membrane filtration.

In certain embodiments, the microbial growth by-products of the subject compositions are biosurfactants selected from, for example, glycolipids (e.g., sophorolipids, cellobiose lipids, rhamnolipids, mannosylerythritol lipids and trehalose lipids), lipopeptides (e.g., surfactin, iturin, fengycin, arthrofactin and lichenysin), flavolipids, fatty acid esters, phospholipids (e.g., cardiolipin, phosphatidylglycerol), and high molecular weight polymers such as lipoproteins, lipopolysaccharide-protein complexes, and polysaccharide-protein-fatty acid complexes.

In certain embodiments, the biosurfactant is a sophorolipid. Sophorolipids are glycolipid biosurfactants produced by, for example, various yeasts of the Starmerella clade. SLP consist of a disaccharide sophorose linked to long chain hydroxy fatty acids. They can comprise a partially acetylated 2-O-β-D-glucopyranosyl-D-glucopyranose unit attached β-glycosidically to 17-L-hydroxyoctadecanoic or 17-L-hydroxy-Δ9-octadecenoic acid. The hydroxy fatty acid is generally 16 or 18 carbon atoms, and may contain one or more unsaturated bonds. Furthermore, the sophorose residue can be acetylated on the 6- and/or 6′-position(s). The fatty acid carboxyl group can be free (acidic or linear form) or internally esterified at the 4″-position (lactonic form).

In some embodiments,, SLP, S. bombicola, and the substrate in which the SLP is produced have been granted GRAS (Generally Regarded as Safe) status by the Food and Drug Administration. In one embodiment, the toxic dose of SLP is >375 mg/kg of body weight.

In certain embodiments, the biosurfactant is a rhamnolipid (RLP). RLP are glycolipids comprising a rhamnose moiety and a 3-(hydroxyalkanoyloxy) alkanoic acid fatty acid tail. Two main classes of rhamnolipids exist, mono-rhamnolipids and di-rhamnolipids, which have one or two rhamnose groups, respectively. The length and degree of branching in the fatty acid tail can also vary between RLP molecules. Most commonly, RLP are produced using the bacterium Pseudomonas aeruginosa; however, P. aeruginosa is a known pathogen to humans and some plants.

In certain embodiments, the biosurfactant is a mannosylerythritol lipid (MEL). MEL are glycolipid biosurfactants comprising either 4-O-β-D-mannopyranosyl-meso-erythritol or 1-O-β-D-mannopyranosyl-meso-erythritol as the hydrophilic moiety, and fatty acid groups and/or acetyl groups as the hydrophobic moiety. One or two of the hydroxyls, typically at the C4 and/or C6 of the mannose residue, can be acetylated. Furthermore, there can be one to three esterified fatty acids, from 8 to 12 carbons or more in chain length.

MEL molecules can be modified, either synthetically or in nature. For example, MEL can comprise different carbon-length chains or different numbers of acetyl and/or fatty acid groups. The molecules can be grouped accordingly: MEL A (di-acetylated), MEL B (mono-acetylated at C4), MEL C (mono-acetylated at C6), MEL D (non-acetylated), tri-acetylated MEL A, and tri-acetylated MEL B/C. Other MEL-like molecules that exhibit similar structures and similar properties can include mannosyl-mannitol lipids (MML), mannosyl-arabitol lipids (MAL), and/or mannosyl-ribitol lipids (MRL). MEL are commonly produced by the yeast Pseudozyma aphidis.

In certain embodiments, the biosurfactant is a lipopeptide. Lipopeptides are oligopeptides synthesized by bacteria using large multi-enzyme complexes. They are frequently used as antibiotic compounds, and exhibit a wide antimicrobial spectrum of action, in addition to surfactant activities. All lipopeptides share a common cyclic structure consisting of a β-amino or β-hydroxy fatty acid integrated into a peptide moiety. Many strains of Bacillus spp. bacteria are capable of producing lipopeptides, for example, Bacillus subtilis and Bacillus amyloliquefaciens.

The most commonly studied family of lipopeptides, the surfactin family, consists of heptapeptides containing a β-hydroxy fatty acid with 13 to 15 carbon atoms. Surfactins are considered some of the most powerful biosurfactants. They are capable of some antiviral activity, as well as antifungal activity, and they exhibit strong synergy when used in combination with another lipopeptide, iturin A. Furthermore, surfactins may also be a key factor in the establishment of stable biofilms, while also inhibiting the biofilm formation of other bacteria, including Gram-negative bacteria.

The fengycin family, which includes plipastatins, comprises decapeptides with a β-hydroxy fatty acid. Fengycins exhibit some unusual properties, such as the presence of ornithine in the peptide portion. They are capable of antifungal activity, although more specific for filamentous fungi.

The iturin family, represented by, e.g., iturin A, mycosubtilin, and bacillomycin, are heptapeptides with a β-amino fatty acid. Iturins also exhibit strong antifungal activity.

Other lipopeptides have been identified, which exhibit a variety of useful characteristics. These include, but are not limited to, kurstakins, arthrofactin, viscosin, glomosporin, amphisin, and syringomycin, to name a few.

Advantageously, in some embodiments, biosurfactants serve as wetting agents, even in hydrophobic soils, due to their ability to lower cohesive and/or adhesive surface tension. This allows water to disperse more evenly and penetrate the soil. Additionally, biosurfactants can serve as biological pesticides, due to, for example, the anti-bacterial, anti-viral, anti-nematodal, and/or anti-fungal capabilities of certain types of biosurfactants. Furthermore, biosurfactants are biodegradable, thereby reducing and/or eliminating the negative side effects resulting from application of chemical wetting agents, pesticides, and/or other chemical agricultural treatments.

The composition can comprise the one or more biosurfactants at a concentration of, for example, 0.001% to 10%, 0.01% to 5%, 0.05% to 2%, and/or from 0.1% to 1% by weight.

To improve or stabilize the effects of the composition, it can be blended with suitable adjuvants and then used as such or after dilution, if necessary. In one embodiment, the composition can comprise glucose (e.g., in the form of molasses), glycerol and/or glycerin, as, or in addition to, an osmoticum substance, to promote osmotic pressure during storage and transport if the product is used in dry form.

The compositions can be used either alone or in combination with other compounds and/or methods for efficiently enhancing soil health and/or plant health, growth and/or yields. For example, in one embodiment, the composition can include and/or can be applied concurrently with nutrients and/or micronutrients for enhancing plant and/or microbe growth, such as magnesium, phosphate, nitrogen, potassium, selenium, calcium, sulfur, iron, copper, and zinc; and/or one or more prebiotics, such as kelp extract, fulvic acid, chitin, humate and/or humic acid. The exact materials and the quantities thereof can be determined by a grower or an agricultural scientist having the benefit of the subject disclosure.

The compositions can also be used in combination with other agricultural compounds and/or crop management systems. In one embodiment, the composition can optionally comprise, or be applied with, for example, natural and/or chemical pesticides, repellants, herbicides, fertilizers, water treatments, non-ionic surfactants and/or soil amendments.

The microbe-based products may be formulated in a variety of ways, including liquid, solids, granular, dust, or slow release products by means that will be understood by those of skill in the art having the benefit of the subject disclosure.

Solid formulations of the invention may have different forms and shapes such as cylinders, rods, blocks, capsules, tablets, pills, pellets, strips, spikes, etc. Solid formulations may also be milled, granulated or powdered. The granulated or powdered material may be pressed into tablets or used to fill pre-manufactured gelatin capsules or shells. Semi solid formulations can be prepared in paste, wax, gel, or cream preparations.

The solid or semi-solid compositions of the invention can be coated using film-coating compounds such as polyethylene glycol, gelatin, sorbitol, gum, sugar or polyvinyl alcohol. This is particularly essential for tablets or capsules. Film coating can protect the handler from coming in direct contact with the active ingredient in the formulations. In addition, a bittering agent such as denatonium benzoate or quassin may also be incorporated in the formulations, the coating or both.

The compositions of the invention can also be prepared in powder formulations and used as-is, or, optionally, filled into pre-manufactured gelatin capsules.

The concentrations of the ingredients in the formulations and application rate of the compositions may be varied widely depending on the soil, plant or area treated, or method of application.

The microbe-based compositions may be used without further stabilization, preservation, and storage. Advantageously, direct usage of these microbe-based compositions preserves a high viability of the microorganisms, reduces the possibility of contamination from foreign agents and undesirable microorganisms, and maintains the activity of the by-products of microbial growth. Furthermore, direct usage of microbial cultures containing cells, nutrients, substrate and/or metabolites increases the number of benefits that the composition provides for agricultural purposes, beyond what benefits are conferred by, for example, metabolites that have been extracted and purified.

In other embodiments, the composition (microbes, growth by-products, growth medium, or combinations thereof) can be placed in containers of appropriate size, taking into consideration, for example, the intended use, the contemplated method of application, the size of the fermentation vessel, and any mode of transportation from microbe growth facility to the location of use. Thus, the containers into which the microbe-based composition is placed may be, for example, from 1 pint to 1,000 gallons or more. In certain embodiments the containers are 1 gallon, 2 gallons, 5 gallons, 25 gallons, or larger

Further components can be added to the composition, for example, buffering agents, carriers, other microbe-based compositions produced at the same or different facility, viscosity modifiers, preservatives, nutrients for microbe growth, tracking agents, biocides, other microbes, surfactants, emulsifying agents, lubricants, solubility controlling agents, pH adjusting agents, preservatives, stabilizers and ultra-violet light resistant agents.

The pH of the microbe-based composition should be suitable for the microorganism and/or microbial growth by-product of interest. In a preferred embodiment, the pH of the composition is about 3.5 to 7.0, about 4.0 to 6.8, or about 5.0 to 6.5.

Optionally, the composition can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C.

In certain embodiments, the compositions of the subject invention have advantages over, for example, biosurfactants alone, including one or more of the following: high concentrations of mannoprotein as a part of a yeast cell wall's outer surface; the presence of beta-glucan in yeast cell walls; and the presence of proteins, polynucleotides, lipids, amino acids, vitamins, biosurfactants and other metabolites in the culture.

### Methods of Enhancing Soil Health and/or Plant Health

In preferred embodiments, the subject invention provides methods for enhancing soil health and/or plant health, wherein a composition comprising one or more microorganisms and/or one or more microbial growth by-products is applied to the soil and/or the plant. In certain embodiments, the growth by-products comprise biosurfactants, such as glycolipids and/or lipopeptides.

In preferred embodiment, a composition according to the subject invention, as described previously, is applied to the soil and/or to the plant. As used herein, “applying” a composition or product, or “treating” an environment refers to contacting a composition or product with a target or site such that the composition or product can have an effect on that target or site. The effect can be due to, for example, microbial growth and/or the action of a metabolite, enzyme, biosurfactant or other growth by-product.

In some embodiments application is performed by spreading a composition of the present invention onto the soil surface. This may be performed using a standard spreader or sprayer device. In some embodiments, a single spreading step may complete the application process, wherein all of the components are included in a single formulation. In other embodiments, which use two- or multiple-part formulations, multiple spreading steps may be used.

In one embodiment, the composition may be rubbed, brushed, or worked into the soil using a mechanical action, for example, by tilling. In still further embodiments, the application of a composition may be subsequently followed by application of a liquid, such as water. The water may be applied as a spray, using standard methods known to one of ordinary skill in the art. Other liquid wetting agents and wetting formulations may also be used.

In certain embodiments, the compositions provided herein are applied to the soil surface without mechanical incorporation. The beneficial effect of the soil application can be activated by rainfall, sprinkler, flood, or drip irrigation, and subsequently delivered to, for example, the roots of plants to influence the root microbiome or facilitate uptake of the microbial product into the vascular system of the crop or plant to which the microbial product is applied. In an exemplary embodiment, the compositions provided herein can be efficiently applied via a center pivot irrigation system or with a spray over the seed furrow.

In some embodiments, the compositions provided herein, either in a dry or in liquid formulation, are applied as a seed treatment or to the soil surface, or to the surface of a plant or plant part (e.g., to the surface of a plant's leaves or roots).

The methods can be utilized in, for example, agricultural fields, pastures, orchards, prairies, plots, and/or forests. The methods can also be utilized in areas containing soil that is significantly uninhabitable by plant life, for example, soils that have been over-cultivated and/or where crop rotation has not been implemented or has been insufficient to retain the soil's fertility; soils that have been polluted by over-treatment with pesticides, fertilizers and/or herbicides; soils with high salinity; soils that have been polluted by dumping, or chemical or hydrocarbon spills; and/or soils in areas damaged by natural or anthropogenic causes, including fire, flooding, pest infestation, development (e.g. commercial, residential or urban building), digging, mining, logging, livestock rearing, and other causes.

Advantageously, the methods can help enhance agricultural yields, even in depleted or damaged soils; restore depleted greenspaces, such as pastures, forests, wetlands and prairies; and restore uncultivatable land so that it can be used for farming, reforestation and/or natural regrowth of plant ecosystems. Additionally, through improved agricultural practices, the methods can help reduce pollution caused by emissions of greenhouse gases.

The applied microbes can be either live (or viable) or inactive at the time of application. In some embodiments, the microbes are in the form of yeast extract and/or another microbial hydrolysate.

The microbial growth by-products can be those produced by the microorganism(s), and/or they can be applied in addition to the growth by-products produced by the microorganism(s) of the composition.

The methods can further comprise adding materials to enhance microbe growth during application (e.g., adding nutrients and/or prebiotics). Thus, live microorganisms can grow in situ and produce the active compounds onsite. Consequently, a high concentration of microorganisms and their growth by-products can be achieved easily and continuously in soil.

In some embodiments, the method comprises applying one or more microbial growth by-products to the soil and/or plant without a microorganism. Specifically, in one embodiment, the method comprises applying a composition comprising crude or pure form glycolipid and/or lipopeptide biosurfactants to the soil and/or plant.

In some embodiments, the method comprises applying an entire microbial culture, comprising inactivated cells in submerged or solid-state fermentation medium. Advantageously, this reduces the amount of waste products produced during production of the subject compositions while increasing efficiency of production by removing the steps of extraction and/or purification of microbial metabolites. Furthermore, inclusion of inactive cells and residual fermentation medium provides rich sources of organic and inorganic nutrients that are essential for supporting soil and/or plant health.

Application of the microbe-based compositions can be performed either alone or in combination with application of other compounds for enhancing soil health and/or plant health. For example, commercial and/or natural fertilizers, pesticides, herbicides and/or other soil amendments can be applied alongside the microbe-based compositions. In certain embodiments, the microbe-based compositions can be used to enhance the effectiveness of the other compounds, for example, by promoting the retention of the compound in soil, or allowing for more uniform dispersal of the compound throughout the soil.

In other applications, desired soil attributes may be obtained by mixing a variety of materials into the soil, including, for example, bone meal, alfalfa, corn gluten, potash, and/or manure from a variety of animals including horses, cows, pigs, chickens, bats, sheep. Other additional elements that can be added include, but are not limited to, mineral nutrients such as magnesium, phosphate, nitrogen, potassium, selenium, calcium, sulfur, iron, copper, and zinc. The exact materials and the quantities thereof can be determined by a soil scientist.

In one embodiment the microbe-based composition of the subject invention is dispersed in soil and/or on a plant while being supported on a carrier. The carrier can be made of materials that can retain microorganisms thereon relatively mildly and thus allow easy release of microorganisms thus proliferated. The carrier is preferably inexpensive and can act as a nutrient source for the microorganisms thus applied, particularly a nutrient source that can be gradually released. Preferred biodegradable carrier materials include cornhusk, sugar industry waste, or any agricultural waste. The water content of the carrier typically varies from 1% to 99% by weight, preferably from 5% to 90% by weight, more preferably from 10% to 85% by weight.

Substances that enhance the growth of microorganisms and the production of biosurfactants may also be added to the microbe-based product and/or the treatment site. These substances include, but not limited to, oil, glycerol, sugar, or other nutrients. For example, a carbon substrate that supports the growth of the biosurfactant-producing microorganisms may be added to the composition or the targeted areas. Biosurfactant producing organisms can grow on the substrate to produce biosurfactants in place.

Although it is not necessary, it may be preferable to spike or amend the carbon substrate with a sufficient amount of specific biosurfactant to initiate the emulsification process and to inhibit or reduce the growth of other competing organisms for the biosurfactant-producing organism.

In certain embodiments, enhancing soil health comprises improving one or more qualities of soil. This can comprise, for example, removing and/or reducing pollutants in the soil, improving the nutrient content and nutrient availability of the soil, improving drainage and/or moisture retention properties of the soil, improving the salinity of the soil, improving the soil microbiome diversity, and/or controlling a soil-borne pest. Other improvements can include adding bulk and/or structure to soils that have been eroded by wind and/or water, as well as preventing and/or delaying erosion of soil by wind and/or water.

In certain embodiments, the methods comprise a step of characterizing the soil type and/or soil health status prior to treating the soil according to the subject methods. Accordingly, the method can also comprise tailoring the composition in order to meet a specific soil type and/or soil health need. Methods of characterizing soils are known in the agronomic arts.

Microbial biomass, whether active or inactive, provides organic matter that improves the physical structure of soils by, for example, adding bulk; helps reduce the erosion of soils by water and wind; and can increase the water retention capacity of soil, particularly porous, sandy soils. Furthermore, active and decaying microbial biomass improves the aeration, and thus water/nutrient infiltration, of heavy and compacted soils.

Other benefits of microbial biomass to soil include providing a nutrient source (e.g. nitrogen, phosphorus, potassium, sulfur, etc.) for plants as well as other soil microorganisms, dissolution of insoluble soil minerals to increase their bioavailability to plant roots due to, for example, favorable cation exchange capacity, regulation of soil temperature, and buffering of pesticide, herbicide, and other heavy metal residues.

In some embodiments, the methods are used for restoring soil health, wherein the soil being treated was once healthy, but deteriorated over some period of time. The restoration may bring the soil back to its previous state of health and/or an enhanced state of health.

In preferred embodiments, the method comprises applying one or more biosurfactants to the soil. Microbial biosurfactants are compounds produced by a variety of microorganisms such as bacteria, fungi, and yeasts. In certain embodiments, they are produced by the microorganisms of the microbe-based composition.

Biosurfactants reduce the surface and interfacial tensions between the molecules of liquids, solids, and gases. Biosurfactants have great potential in soil biology because they are biodegradable, have low toxicity, are effective in solubilizing and degrading insoluble compounds in soil and can be produced using renewable resources. Furthermore, biosurfactants can also have powerful emulsifying and demulsifying properties, and can be used to obtain soil wettability and to achieve even distribution of fertilizers, nutrients, and water in the soil.

Biosurfactants are unique in that they are produced via microbial fermentation but have those properties possessed by chemical surfactants in addition to other attributes not possessed by their synthetic analogs. Biosurfactants decrease the tendency of water to pool, they improve the adherence or wettability of surfaces, resulting in more thorough hydration of soil, and they reduce the volume of water that might otherwise drain or escape below the root zone via micro-channels formed by drip and micro-irrigation systems. This wettability also promotes better root system health, as there are fewer zones of desiccation (or extreme dryness) inhibiting proper root growth and better availability of applied nutrients as chemical and micro-nutrients are more thoroughly made available and distributed.

The more uniform distribution of water in soil made possible by enhanced wettability also prevents water from accumulating or getting trapped above optimal penetration levels, thereby mitigating anaerobic conditions that inhibit the free exchange of oxygen and carbon. Once a biosurfactant is applied, a more porous or breathable soil is established. The combination of a properly hydrated and aerated soil also increases the susceptibility of soil pests and pathogens (such as nematodes and soil borne fungi and their spores) to pest control agents. Additionally, some biosurfactants have antibacterial, antiviral, and/or antifungal properties. Thus, biosurfactants can be used for a wide range of useful applications, including disease and pest control.

In certain embodiments, the method results in removal and/or reduction of pollutants from soil, including remediation of soils contaminated with hydrocarbons. In some embodiments, the pollutants are degraded directly by the applied microorganisms of the composition. In some embodiments, the growth by-products of the microorganisms, e.g., biosurfactants, facilitate degradation of the pollutants, and can chelate and form a complex with ionic and nonionic metals to release them from the soil. Soil pollutants include, for example, residual fertilizers, pesticides, herbicides, fungicides, hydrocarbons, chemicals (e.g., dry cleaning treatments, urban and industrial wastes), benzene, toluene, ethylbenzene, xylene, and heavy metals.

In some embodiments, the biosurfactants serve as emulsifiers, increasing the oil-water interface of hydrocarbon pollutants by forming stable microemulsions with them. The result is an increase in the mobility and bioavailability of the pollutants for decomposing microorganisms.

The methods can further comprise supplying oxygen and/or nutrients to the microorganisms by circulating aqueous solutions through the soils, thus stimulating the applied microorganisms, as well as naturally-occurring soil microorganisms, to degrade the pollutants and/or produce pollutant-degrading growth by-products. In some embodiments, the polluted soil is combined with nonhazardous organic amendments such as manure or agricultural wastes. The presence of these organic materials supports the development of a rich microbial population and elevated temperature characteristics of composting. Thus, the rate of bioremediation can be increased.

In certain embodiments, the method results in improvement in the soil nutrient content and availability to plant roots. Biosurfactants enhance mobility of metals in soil to plants. Furthermore, microbial biomass, including live and inactive biomass, provide sources of nutrients, such as nitrogen, phosphorous and potassium (NPK), amino acids, vitamins, proteins and lipids.

In certain embodiments, the method results in improved drainage and/or moisture retention properties of soil of dry, waterlogged, porous, depleted, compacted soils and/or combinations thereof. In one embodiment, the method can be used for improving the drainage and/or dispersal of water in waterlogged soils. In one embodiment, the method can be used for improving water retention in dry soil. Advantageously, in some embodiments, the methods help reduce agricultural water consumption, even in drought.

In one embodiment, the method is useful for improving water retention in sandy and hydrophobic soils, which have high drainage. Biosurfactants wet these soils by lowering the cohesive and/or adhesive surface tension, allowing the water to spread more evenly and penetrate the soil.

In certain embodiments, the method results in improved salinity of soil by reducing the salt content. Saline soils contain sufficient neutral soluble salts to adversely affect the growth of most crop plants. Soluble salts most commonly present are the chlorides and sulfates of sodium, calcium and magnesium. Nitrates may be present rarely, while many saline soils contain appreciable quantities of gypsum (CaSO₄, 2H₂O).

When leached with low-salt water, some saline soils tend to disperse, resulting in low permeability to water and air, particularly when the soils are heavy clays. The presence of microorganisms and/or biosurfactants improves the mobility of salts and/or ions, thereby facilitating drainage of salts into depths below plant root zones.

In certain embodiments, the methods can also help improve soil microbiome diversity by promoting colonization of the soil and plant roots growing therein with beneficial soil microorganisms. Growth of nutrient-fixing microbes, such as rhizobium and/or mycorrhizae, can be promoted, as well as other endogenous and applied microbes, thereby increasing the number of different species within the soil microbiome.

In some embodiments, the methods are used for controlling above-ground and below-ground pests. In some embodiments, the method can be useful for controlling pests such as arthropods, nematodes, protozoa, bacteria, fungi, and/or viruses. In some embodiments, the method can be useful for modulating a plant's immune system to activate the plant's innate defenses against pests.

In some embodiments, the methods are used for stimulating the growth of plants, enhancing plant health and/or yields, and/or improving the plants' ability to outcompete weeds and other detrimental plants.

### Growth of Microbes

The subject invention utilizes methods for cultivation of microorganisms and production of microbial metabolites and/or other by-products of microbial growth. The subject invention further utilizes cultivation processes that are suitable for cultivation of microorganisms and production of microbial metabolites on a desired scale. These cultivation processes include, but are not limited to, submerged cultivation/fermentation, solid state fermentation (SSF), and modifications, hybrids and/or combinations thereof.

As used herein “fermentation” refers to cultivation or growth of cells under controlled conditions. The growth could be aerobic or anaerobic. In certain embodiments, the microorganisms are grown using SSF and/or modified versions thereof.

In one embodiment, the subject invention provides materials and methods for the production of biomass (e.g., viable cellular material), extracellular metabolites (e.g. small molecules and excreted proteins), residual nutrients and/or intracellular components (e.g. enzymes and other proteins).

The microbe growth vessel used according to the subject invention can be any fermenter or cultivation reactor for industrial use. In one embodiment, the vessel may have functional controls/sensors or may be connected to functional controls/sensors to measure important factors in the cultivation process, such as pH, oxygen, pressure, temperature, humidity, microbial density and/or metabolite concentration.

In a further embodiment, the vessel may also be able to monitor the growth of microorganisms inside the vessel (e.g., measurement of cell number and growth phases). Alternatively, a daily sample may be taken from the vessel and subjected to enumeration by techniques known in the art, such as dilution plating technique. Dilution plating is a simple technique used to estimate the number of organisms in a sample. The technique can also provide an index by which different environments or treatments can be compared.

In one embodiment, the method includes supplementing the cultivation with a nitrogen source. The nitrogen source can be, for example, potassium nitrate, ammonium nitrate ammonium sulfate, ammonium phosphate, ammonia, urea, and/or ammonium chloride. These nitrogen sources may be used independently or in a combination of two or more.

The method can provide oxygenation to the growing culture. One embodiment utilizes slow motion of air to remove low-oxygen containing air and introduce oxygenated air. In the case of submerged fermentation, the oxygenated air may be ambient air supplemented daily through mechanisms including impellers for mechanical agitation of liquid, and air spargers for supplying bubbles of gas to liquid for dissolution of oxygen into the liquid.

The method can further comprise supplementing the cultivation with a carbon source. The carbon source is typically a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as soybean oil, canola oil, rice bran oil, olive oil, corn oil, sesame oil, and/or linseed oil; etc. These carbon sources may be used independently or in a combination of two or more.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, and microelements can be included, for example, in the form of flours or meals, such as corn flour, or in the form of extracts, such as yeast extract, potato extract, beef extract, soybean extract, banana peel extract, and the like, or in purified forms. Amino acids such as, for example, those useful for biosynthesis of proteins, can also be included.

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, sodium chloride, calcium carbonate, and/or sodium carbonate. These inorganic salts may be used independently or in a combination of two or more.

In some embodiments, the method for cultivation may further comprise adding additional acids and/or antimicrobials in the medium before, and/or during the cultivation process. Antimicrobial agents or antibiotics are used for protecting the culture against contamination.

Additionally, antifoaming agents may also be added to prevent the formation and/or accumulation of foam, in the case of submerged cultivation.

The pH of the mixture should be suitable for the microorganism of interest. Buffers, and pH regulators, such as carbonates and phosphates, may be used to stabilize pH near a preferred value. When metal ions are present in high concentrations, use of a chelating agent in the medium may be necessary.

The microbes can be grown in planktonic form or as biofilm. In the case of biofilm, the vessel may have within it a substrate upon which the microbes can be grown in a biofilm state. The system may also have, for example, the capacity to apply stimuli (such as shear stress) that encourages and/or improves the biofilm growth characteristics.

In one embodiment, the method for cultivation of microorganisms is carried out at about 5° to about 100° C., preferably, 15 to 60° C., more preferably, 25 to 50° C. In a further embodiment, the cultivation may be carried out continuously at a constant temperature. In another embodiment, the cultivation may be subject to changing temperatures.

In one embodiment, the equipment used in the method and cultivation process is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the medium may be pasteurized or, optionally, no heat at all added, where the use of low water activity and low pH may be exploited to control undesirable bacterial growth.

In one embodiment, the subject invention further provides a method for producing microbial metabolites such as, for example, biosurfactants, enzymes, proteins, ethanol, lactic acid, beta-glucan, peptides, metabolic intermediates, polyunsaturated fatty acid, and lipids, by cultivating a microbe strain of the subject invention under conditions appropriate for growth and metabolite production; and, optionally, purifying the metabolite. The metabolite content produced by the method can be, for example, at least 20%, 30%, 40%, 50%, 60%, 70%, 80%, or 90%.

The microbial growth by-product produced by microorganisms of interest may be retained in the microorganisms or secreted into the growth medium. The medium may contain compounds that stabilize the activity of microbial growth by-product.

The biomass content of the fermentation medium may be, for example, from 5 g/l to 180 g/l or more, or from 10 g/l to 150 g/l.

The cell concentration may be, for example, at least 1×10⁶ to 1×10¹², 1×10⁷ to 1×10¹¹, 1×10⁸ to 1×10¹⁰, or 1×10⁹ CFU/ml.

The method and equipment for cultivation of microorganisms and production of the microbial by-products can be performed in a batch, a quasi-continuous process, or a continuous process.

In one embodiment, all of the microbial cultivation composition is removed upon the completion of the cultivation (e.g., upon, for example, achieving a desired cell density, or density of a specified metabolite). In this batch procedure, an entirely new batch is initiated upon harvesting of the first batch.

In another embodiment, only a portion of the fermentation product is removed at any one time. In this embodiment, biomass with viable cells, spores, conidia, hyphae and/or mycelia remains in the vessel as an inoculant for a new cultivation batch. The composition that is removed can be a cell-free medium or contain cells, spores, or other reproductive propagules, and/or a combination of thereof. In this manner, a quasi-continuous system is created.

Advantageously, the method does not require complicated equipment or high energy consumption. The microorganisms of interest can be cultivated at small or large scale on site and utilized, even being still-mixed with their media.

Advantageously, the microbe-based products can be produced in remote locations. The microbe growth facilities may operate off the grid by utilizing, for example, solar, wind and/or hydroelectric power.

### Preparation of Microbe-based Products

One microbe-based product of the subject invention is simply the fermentation medium containing the microorganisms and/or the microbial metabolites produced by the microorganisms and/or any residual nutrients. The product of fermentation may be used directly without extraction or purification. If desired, extraction and purification can be easily achieved using standard extraction and/or purification methods or techniques described in the literature.

The microorganisms in the microbe-based products may be in an active or inactive form, or in the form of vegetative cells, reproductive spores, conidia, mycelia, hyphae, or any other form of microbial propagule. The microbe-based products may also contain a combination of any of these forms of a microorganism. In preferred embodiments, the microorganisms and/or propagules are inactivated.

In one embodiment, different strains of microbe are grown separately and then the cultures are mixed together to produce the microbe-based product. The microbes can, optionally, be blended with the medium in which they are grown and dried prior to mixing.

In one embodiment, the different strains are not mixed together, but are applied to soil as separate microbe-based products.

The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

In some embodiment, however, biosurfactants and/or other metabolites can be extracted from the culture, and optionally, purified. In further embodiments, two or more extracted biosurfactants and/or other metabolites can be mixed together to form a biosurfactant cocktail.

Upon harvesting the microbe-based composition from the growth vessels, further components can be added as the harvested product is placed into containers or otherwise transported for use. The additives can be, for example, buffers, carriers, other microbe-based compositions produced at the same or different facility, viscosity modifiers, preservatives, nutrients for microbe growth, surfactants, emulsifying agents, lubricants, solubility controlling agents, tracking agents, solvents, biocides, antibiotics, pH adjusting agents, chelators, stabilizers, ultra-violet light resistant agents, other microbes and other suitable additives that are customarily used for such preparations.

In one embodiment, buffering agents including organic and amino acids or their salts, can be added. Suitable buffers include citrate, gluconate, tartarate, malate, acetate, lactate, oxalate, aspartate, malonate, glucoheptonate, pyruvate, galactarate, glucarate, tartronate, glutamate, glycine, lysine, glutamine, methionine, cysteine, arginine and a mixture thereof. Phosphoric and phosphorous acids or their salts may also be used. Synthetic buffers are suitable to be used but it is preferable to use natural buffers such as organic and amino acids or their salts listed above.

In a further embodiment, pH adjusting agents include potassium hydroxide, ammonium hydroxide, potassium carbonate or bicarbonate, hydrochloric acid, nitric acid, sulfuric acid or a mixture.

The pH of the microbe-based composition should be suitable for the microorganism(s) of interest. In a preferred embodiment, the pH of the composition is about 3.5 to 7.0, or about 4.0 to 6.8, or about 5.0 to 6.5.

In one embodiment, additional components such as an aqueous preparation of a salt, such as sodium bicarbonate or carbonate, sodium sulfate, sodium phosphate, sodium biphosphate, can be included in the formulation.

In one embodiment, glucose, glycerol and/or glycerin can be added to the microbe-based product to serve as, for example, an osmoticum during storage and transport. In one embodiment, molasses can be included.

In one embodiment, prebiotics can be added to and/or applied concurrently with the microbe-based product to enhance microbial growth. Suitable prebiotics, include, for example, kelp extract, fulvic acid, chitin, humate and/or humic acid. In a specific embodiment, the amount of prebiotics applied is about 0.1 L/acre to about 0.5 L/acre, or about 0.2 L/acre to about 0.4 L/acre.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C.

### Local Production of Microbe-Based Products

In certain embodiments of the subject invention, a microbe growth facility produces fresh, high-density microorganisms and/or microbial growth by-products of interest on a desired scale. The microbe growth facility may be located at or near the site of application. The facility produces high-density microbe-based compositions in batch, quasi-continuous, or continuous cultivation.

The microbe growth facilities of the subject invention can be located at the location where the microbe-based product will be used (e.g., a citrus grove). For example, the microbe growth facility may be less than 300, 250, 200, 150, 100, 75, 50, 25, 15, 10, 5, 3, or 1 mile from the location of use.

Because the microbe-based product can be generated locally, without resort to the microorganism stabilization, preservation, storage and transportation processes of conventional microbial production, a much higher density of microorganisms can be generated, thereby requiring a smaller volume of the microbe-based product for use in the on-site application or which allows much higher density microbial applications where necessary to achieve the desired efficacy. This allows for a scaled-down bioreactor (e.g., smaller fermentation vessel, smaller supplies of starter material, nutrients and pH control agents), which makes the system efficient and can eliminate the need to stabilize cells or separate them from their culture medium. Local generation of the microbe-based product also facilitates the inclusion of the growth medium in the product. The medium can contain agents produced during the fermentation that are particularly well-suited for local use.

Locally-produced high density, robust cultures of microbes are more effective in the field than those that have remained in the supply chain for some time. The microbe-based products of the subject invention are particularly advantageous compared to traditional products wherein cells have been separated from metabolites and nutrients present in the fermentation growth media. Reduced transportation times allow for the production and delivery of fresh batches of microbes and/or their metabolites at the time and volume as required by local demand.

The microbe growth facilities of the subject invention produce fresh, microbe-based compositions, comprising the microbes themselves, microbial metabolites, and/or other components of the medium in which the microbes are grown. If desired, the compositions can have a high density of vegetative cells or propagules, or a mixture of vegetative cells and propagules.

Advantageously, the compositions can be tailored for use at a specified location. In one embodiment, the microbe growth facility is located on, or near, a site where the microbe-based products will be used (e.g., a citrus grove).

Advantageously, these microbe growth facilities provide a solution to the current problem of relying on far-flung industrial-sized producers whose product quality suffers due to upstream processing delays, supply chain bottlenecks, improper storage, and other contingencies that inhibit the timely delivery and application of, for example, a viable, high cell-count product and the associated medium and metabolites in which the cells are originally grown.

The microbe growth facilities provide manufacturing versatility by their ability to tailor the microbe-based products to improve synergies with destination geographies. Advantageously, in preferred embodiments, the systems of the subject invention harness the power of naturally-occurring local microorganisms and their metabolic by-products to improve agricultural production.

The cultivation time for the individual vessels may be, for example, from 1 to 7 days or longer. The cultivation product can be harvested in any of a number of different ways.

Local production and delivery within, for example, 24 hours of fermentation results in pure, high cell density compositions and substantially lower shipping costs. Given the prospects for rapid advancement in the development of more effective and powerful microbial inoculants, consumers will benefit greatly from this ability to rapidly deliver microbe-based products.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1—Preparation of Compositions Compromising Sophorolipids Using Submerged Fermentation

A mixture of sophorolipids is synthesized by fermentation of S. bombicola in a fermentation medium containing 100 g/L glucose, 10 g/L yeast extract, 1 g/L urea, 100 ml/L canola oil in water, and 0.01 to 0.5 g/L of microelements. All components of the fermentation medium are GRAS. After 5-7 days of fermentation, approximately 500 g/L of sophorolipid precipitates as a brown layer at the bottom of the fermentation vessel.

The sophorolipid layer is collected and diluted 4-fold to a SLP concentration of 125 g/L. The SLP does not require purification using toxic solvents, such as ethyl acetate, because every component of the resulting crude biosurfactant is beneficial or non-harmful for agricultural purposes.

The percentage of SLP in the crude product is 65 to 90%, with insignificant amounts of residual glucose and fatty acid. The ratio of lactonic to acidic form SLP is about 70:30. The surface tension reduction of such a product is ≤35 mN/m at CMC ≤100 ppm. The pH can be adjusted to, e.g., 6.5-7.0, using sodium hydroxide.

### Example 2—SLP For Improving Soil Wettability, Fertility, Salinity and Osmotic Pressure

### Soil Wettability

Soil hydrophobicity causes water to collect on the soil surface rather than infiltrate into the ground. Soil water repellency can be caused by the presence of hydrophobic coatings on soil particles. For example, wild fires can cause soil water repellency due to waxy substances that coat soil particles, produced by the burning of certain plant material. This increases water repellency, runoff of water and nutrients, and erosion in post-burn sites.

In one embodiment, treatment of hydrophobic soil with a composition comprising hydrophobic SLP (e.g., lactonic SLP and/or di- or mono-acetylated acidic SLP) reduces the water-repellency of hydrophobic soil's, allowing increased penetration of the water into the soil and more even dispersion of water and nutrients in the soil.

### Soil Fertility

In one embodiment, a composition comprising hydrophobic SLP (e.g., lactonic SLP and/or di- or mono-acetylated acidic SLP) can enhance the adsorption of nutrients from soil by plant roots (e.g., NPK, boron, chlorine, cobalt, copper, iron, manganese, magnesium, molybdenum, sulfur, zinc, calcium, nickel, silicon and sodium), thus promoting plant growth and higher crop yields. Additionally, by increasing the wettability of soils, more even dispersion of the nutrients throughout the soil can also be achieved, thereby increasing the availability of nutrients to plant roots.

### Soil Salinity

Saline soils cannot be reclaimed by chemical amendments, conditioners or fertilizers, but instead by leaching salts from the plant root zone. In one embodiment, a composition comprising hydrophobic SLP (lactonic SLP and/or di- or mono-acetylated acidic SLP) increases the wettability, dispersion and penetration of water in the soil. Thus, over time, the composition “pushes” salts to greater depths within the soil and under the rhizosphere, so that the soil layers closer to the surface can be used for agricultural purposes.

### Osmotic Pressure

Osmotic pressure occurs when solutions of different ion or solute concentrations are separated by a semi-permeable membrane. Random motion of water and solute molecules create a net movement of water to the compartment with higher solute concentration, until equilibrium is reached. This net movement by concentration differences is called diffusion.

When soil moisture content is low, the osmotic pressure of the tissue fluids in both roots and above-ground portions of plants increases over time, which results in a lower rate of vegetative growth, modifications in stomatal opening, a depletion in starch reserves, a decrease in apparent photosynthesis, and an increase in respiration.

In one embodiment, a composition comprising hydrophobic SLP (e.g. lactonic SLP and/or di- or mono-acetylated acidic SLP) increases the wettability, dispersion and penetration of water in the soil. Thus, over time, the osmotic pressure of plant tissue will reduce and/or approach equilibrium, which allows the plants to grow faster and larger, and in some instances, outcompete other invasive or weedy plants.

### Example 3—SLP for Pest Control

SLP can have strong antibacterial, antifungal, and/or antiviral capabilities. In one embodiment, the effective SLP concentration for biopesticide activity is 0.009 to 10 mg/L, but in most cases it does not exceed 3 mg/L.

SLP is effective against a number of bacterial plant pathogens such as, for example, Acidovorax carotovorum, Erwinia amylovora, Pseudomonas cichorii, Pseudomonas syringae, Pectobacterium carotovorum, Ralstonia solanacearum, Xylella fastidiosa and Xanthomonas campestris;

against fungal plant pathogens such as, for example, Alternaria spp., Aspergillus spp., Fusarium spp., Penicillium spp., Penicillium spp., Saccharomyces spp., Cladosporium spp., Gloeophyllum spp. and Schizophyllum spp., Hemileia spp. (e.g., H. vastatrix), Botrytis cineria and Phytopthora spp.;

against some plant viruses, such as herpesviruses; and against some nematodes.

In one embodiment, the composition comprises lactonic SLP. Lactonic SLP can be useful for lysis of cell walls, thus making the composition favorable for antibacterial and antifungal applications.

In one embodiment, the composition comprises acidic SLP, which is more favorable for antiviral applications.

In one embodiment, the composition comprises about 70% lactonic SLP and 30% linear SLP, providing for effectiveness against a variety of plant pathogens including bacteria, fungi, viruses and nematodes.

### Example 4—Preparation of Compsitions Compromising Rhamnolipids Using Solid-State Fermentation

The highest accumulation of rhamnolipids (RLP) has been shown by submerged cultivation of Pseudomonas aeruginosa, which is an opportunistic pathogen. The pathogenic nature of P. aeruginosa also limits production of RLP, due to the risks to workers of exposure to the microbe.

In certain embodiments, the subject invention utilizes a non-pathogenic bacterium, Burkholderia thailandensis, for producing RLP. A solid-state, or matrix, fermentation method is utilized, wherein corn bran is used as the solid substrate. Glycerol, yeast extract, potato dextrose and some small amounts of trace elements are mixed in with the corn bran.

The substrate is inoculated with the B. thailandensis, and cultivated for 7 to 8 days, and optionally, dried. This will produce about 20 to 30 g of RLP per kilogram of dried culuter/substrate.

Advantageously, the method does not require any additional extraction or purification steps, apart from drying and inactivating the bacterial cells, because, in some embodiments, the resulting mass comprising corn bran, Burkholderia cells and RLP is more beneficial for soil and plants than RLP alone. In certain embodiments, this is due to the vitamins, amino acids, proteins, and minerals present in corn bran (e.g., betaine, choline, folate, folic acid, niacin, riboflavin, vitamin A, carotene, B vitamins, vitamin K, calcium copper, iron, manganese, magnesium, phosphorus, selenium, potassium, zinc, and others; as well as the nutrients present in inactivated bacterial cells (e.g., organic nitrogen, carbon, sulfur, phosphorus, potassium, copper, magnesium, and others).

### Example 5—RLP For Improving Soil Fertility

Zinc, copper, iron, manganese and other trace elements present in soils and fertilizer compounds are often difficult for plant roots to absorb. Though the use of chelating agents, such as ethylenediaminetetraacetic acid (EDTA) and diethylenetriamene pentaacetate (DTPA), are commonly used to increase the persistence of trace elements in soil, metal-EDTA and DTPA complexes are not readily absorbed by plant roots, which limits fertilizer efficacy.

In one embodiment, a composition comprising RLP can help improve the fertility of soil by facilitating absorption of zinc and other trace elements in the soil by plant roots. In one embodiment, the RLP forms a lipophilic complex with zinc, copper, iron and/or manganese, which can improve the bioavailability of these trace elements to the plant roots.

### Example 6—RLP For Removal of Soil Pollutants

The productivity of agricultural land can be affected by the presence of organic and inorganic pollutants that impart abiotic stress on crop plants.

In one embodiment, a composition comprising from 0.05% to 0.5%, or about 0.1% RLP by volume enhances removal of arsenic and/or heavy metal pollutants from soil by acting as a chelating agent that can form a complex with these materials and facilitate their release and/or drainage from plant root zone soil layers. Polluted soils can thus be treated in this way in order for the land to be usable for agricultural purposes.

### Example 7—RLP for Pest Control

In one embodiment a composition comprising about 0.04 to 35 mg/L, or 0.1 to 25 mg/L, or 0.5 to 15 mg/L of RLP can be useful for pest control in two ways.

First, in some embodiments, the composition can have a direct effect on pests due to the pesticidal properties of RLP. Rhamnolipids are active against bacteria, including for example, Pseudomonas aeruginosa, Enterobacter aerogenes, Serratia marcescens, Klebsiella pneumonia, Micrococcus spp., Streptococcus spp., Staphylococcus spp. and Bacillus spp., Xylella spp.; and against certain fungi, including for example, Botrytis spp. (e.g., B. cinerea), Rhizoctonia spp., Pythium spp., Phytophtora spp. and Plasmopara spp., Mucor miehei and Neurospora crassa.

Rhamnolipids are also active against certain arthropods, for example, Aedes aegypti larvae, green peach Aphid (Myzus persicae), arachnids, grasshoppers and box-elder bugs.

Second, a composition comprising RLP can also have an indirect effect for pest control, wherein the RLP help modulate the immune system of a plant to elicit defense responses and induce disease resistance against pests (e.g., hemibiotrophic bacteria), as well as other biotic and/or abiotic stressors.

### Example 8—Preparation of Compositions Compromising Mannosylerythritol Lipids Using Solid State Fermentation

In one embodiment, MEL are produced using Pseudozyma aphidis in a solid-state reactor. The solid substrate comprises a mixture of soybean, yeast extract, erythritol and some insignificant amounts of trace elements.

Upon reaching a desired cell count and/or metabolite concentration in the solid-state reactor, the entire culture is dried to produce a product comprising MEL, substrate and inactivated Pseydozyma cells. This product is more beneficial for soil and plants than MEL alone because both soybeans and inactivated yeast cells are good sources of nutrients such as, for example, organic nitrogen, phosphorus and potassium.

### Example 9—MEL for Pest Control

Compositions comprising MEL are advantageous for pest control given the wide range of pesticidal activity of MEL. In certain embodiments, MEL can be used for controlling microbial pests of:

soybeans and/or canola, including, for example, Phytophthora megasperma sp. glycinea, Macrophomina phaseolina, Rhizoctonia solani, Sclerotinia sclerotiorum, Fusarium spp. (e.g., F. oxysporum, F. semitectum, F. roseum, F. solani), Diaporthe spp. (e.g., D. phaseolorum var. sojae (Phomopsis sojae), D. phaseolorum var. caulivora), Alternaria spp. (e.g., A. brassicae, A. alternate), Sclerotium rolfsii, Cercospora spp. (e.g., C. kikuchii, C. sojina), Pythium spp. (e.g. P. aphanidermatum, P. ultimum, P. debaryanum), Peronospora spp. (e.g., P. manshurica, P. parasitica), Colletotrichum dernatium (C. truncatum), Corynespora cassiicola, Septoria glycines, Phyllosticta sojicola, Pseudomonas syringae p.v. glycinea, Xanthomonas campestris p.v. phaseoli, Microsphaera diffusa, Phialophora gregata, Glomerella glycines, Phakopsora pachyrhizi, Heterodera glycines Leptosphaeria maculans, Mycosphaerella brassiccola, Albugo candida, Soybean mosaic virus, Tobacco Ring spot virus, Tobacco Streak virus, and Tomato spotted wilt virus;

alfalfa, including, for example, Clavibacter michiganensis subsp. insidiosum, Pythium spp. (e.g., P. ultimum, P. irregulare, P. splendens, P. debaryanum, P. aphanidermatum), Phytophthora megasperma, Peronospora trifoliorum, Phoma medicaginis var. medicaginis, Cercospora medicaginis, Pseudopeziza medicaginis, Leptotrochila medicaginis, Fusarium spp., Xanthomonas campestris p.v. alfalfae, Aphanomyces euteiches, and Stemphylium spp. (e.g., S. herbarum, S. alfalfa);

wheat, including, for example, Pseudomonas spp. (e.g., P syringae p.v. atrofaciens, P. syringae p.v. syringae), Urocystis agropyri, Xanthomonas campestris p.v. translucens, Alternaria alternata, Cladosporium herbarum, Fusarium spp. (e.g., F. graminearum, F. avenaceum, F. culmorum), Ascochyta tritici, Cephalosporium gramineum, Collotetrichum graminicola, Erysiphe graminis fsp. tritici, Puccinia spp. (e.g., P. recondita fsp. tritici, P. striiformis, P. graminis fsp. tritici), Pyrenophora tritici-repentis, Septoria spp. (e.g., S. nodorum, S. tritici, S. avenae), Pseudocercosporella herpotrichoides, Rhizoctonia spp. (e.g., R. solani, R. cerealis), Gaeumannomyces graminis var. tritici, Pythium spp. (e.g., P. aphanidermatum, P. arrhenomanes, P. ultimum, P. arrhenomanes, P. gramicola, P. aphanidermatum), Bipolaris sorokiniana, Claviceps purpurea, Tilletia spp. (e.g., T. tritici, T. laevis, T. indica), Ustilago tritici, Barley Yellow Dwarf Virus, Brome Mosaic Virus, Soil Borne Wheat Mosaic Virus, Wheat Streak Mosaic Virus, Wheat

Spindle Streak Virus, American Wheat Striate Virus, High Plains Virus, and European wheat striate virus;

sunflowers, including for example, Plasmophora halstedii, Sclerotinia sclerotiorum, Septoria helianthi, Phomopsis helianthi, Alternaria spp. (e.g., A. helianthi, A. zinnia), Botrytis cinerea, Phoma macdonaldii, Macrophomina phaseolina, Erysiphe cichoracearum, Rhizopus spp. (e.g., R. oryzae, R. arrhizus, R. stolonifera), Puccinia helianthi, Verticillium dahliae, Erwinia carotovorum p.v. carotovora, Cephalosporium acremoniwn, Phytophthora cryptogea, Albugo tragopogonis, and Aster Yellows;

corn, including, for example, Fusarium spp. (e.g., F. moniliforme var. subglutinans, F. verticilloides, F. moniliforme, Gibberella zeae (F. graminearum)), Stenocarpella maydis (Diplodia maydis), Pythium spp. (e.g., P. irregulare, P.debaryanum, P. graminicola, P. splendens, P. ultimum, P. aphanidermatum), Aspergillus flavus, Bipolaris maydis O, T (Cochliobolus heterostrophus), Helminthosporium spp. (e.g., H. carbonum I, II & III (Cochliobolus carbonum), H pedicellatum), Exserohilum turcicum I, II & III, Physoderma maydis, Phyllosticta maydis, Kabatiella maydis, Cercospora sorghi, Ustilago maydis, Puccinia spp. (e.g., P. sorghi, P. polysora), Macrophomina phaseolina, Penicillium oxalicum, Nigrospora oryzae, Cladosporium herbarum, Curvularia spp. (e.g., C. lunata, C. inaequalis, C. pallescens), Clavibacter michiganense subsp. nebraskense, Trichoderma viride, Claviceps sorghi, Pseudomonas avenae, Erwinia spp. (e.g., E. carotovora, E. stewartii, E. chrysanthemi pv. Zea), Diplodia macrospora, Sclerophthora macrospora, Peronosclerospora spp. (e.g., P. sorghi, P. philippinensis, P. maydis, P. sacchari), Sphacelotheca reiliana, Physopella zeae, Cephalosporium spp. (e.g., C. maydis, C. acremonium), Maize Dwarf Mosaic Virus A & B, Wheat Streak Mosaic Virus, Maize Chlorotic Dwarf Virus, Corn stunt spiroplasma, Maize Chlorotic Mottle Virus, High Plains Virus, Maize Mosaic Virus, Maize Rayado Fino Virus, Maize Streak Virus, Maize Stripe Virus, and Maize Rough Dwarf Virus;

sorghum, including, for example, Exserohilum turcicum, Colletotrichum graminicola (Glomerella graminicola), Cercospora sorghi, Gloeocercospora sorghi, Ascochyta sorghina, Pseudomonas spp. (e.g., P. avenae (P. alboprecipitans), P. syringae p.v. syringae, P. andropogonis), Xanthomonas campestris p.v. holcicola, Puccinia purpurea, Macrophomina phaseolina, Periconia circinata, Fusarium spp. (e.g., P. moniliforme, F. graminearum, F. oxysporum), Alternaria alternata, Bipolaris sorghicola, Helminthosporium sorghicola, Curvularia lunata, Phoma insidiosa, Ramulispora spp. (e.g., R. sorghi, R. sorghicola), Phyllachara sacchari, Sporisorium spp. (e.g., S. reilianum (Sphacelotheca reiliana), S. sorghi), Sphacelotheca cruenta, Claviceps sorghi, Rhizoctonia solani, Acremonium strictum, Sclerophthora macrospora, Peronosclerospora spp. (P. sorghi, P. philippinensis), Sclerospora graminicola, Pythium spp. (P. arrhenomanes, P. graminicola), Sugarcane mosaic H, and Maize Dwarf Mosaic Virus A & B;

and rice, including, for example, Magnaporthe grisea and Rhizoctonia solani.

In certain embodiments, compositions comprising MEL can be used for controlling nematode pests, including, for example, Ditylenchus dipsaci, Aphelenchoides ritzemabosi, Heterodera spp. (e.g., H. trifolii, H. schachtii), Xiphinema americanum, Pratylenchus spp., (e.g., P. vulnus, P. neglectus, P. penetrans, P. hamatus), Longidorus spp., Rotylenchulus spp., Meloidogyne spp., (e.g., M. arenaria, M. chitwoodi, M. hapla, M. incognita, M. javanica), Helicotylenchus spp., Paratrichodorus spp., Tylenchorhynchus spp., (e.g., T. semipenetrans), Belonolaimus longicaudatus, and Criconemella xenoplax,

In certain embodiments, compositions comprising MEL can be used for controlling arthropod pests, including, for example, Acalymma, Acleris variegana, African armyworm, Africanized bee, Agromyzidae, Agrotis munda, Agrotis porphyricollis, Aleurocanthus woglumi, Aleyrodes proletella, Anasa tristis, Anisoplia austriaca, Anthonomus pomorum, Anthonomus signatus, Aonidiella aurantii, aphid, Aphis fabae, Aphis gossvpii, apple maggot, Argentine ant, army cutworm, Arotrophora arcuatalis, Asterolecanium coffeae, Australian plague locust, Bactericera cockerelli, Bactrocera correcta, Bagrada hilaris, banded hickory borer, Banksia boring moth, beet armyworm, bogong moth, boll weevil, Brevicoryne brassicae, Brown locust, brown marmorated stink bug, brown planthopper, cabbage moth, cabbage worm, Callosobruchus maculatus, cane beetle, carrot fly, Cecidomyiidae, Ceratitis capitata, cereal leaf beetle, Chlorops pumilionis, citrus long-horned beetle, Coccus viridis, codling moth, coffee borer beetle, Colorado potato beetle, confused flour beetle, Crambus, cucumber beetle, Curculio nucum, cutworm, dark sword-grass, date stone beetle, Delia (genus), Delia antiqua, Delia floralis, Delia radicum, desert locus, Diabrotica, diamondback moth, Diaphania indica, Diaphania nitidalis, Diaphorina citri, Diaprepes abbreviatus, differential grasshopper, Dociostaurus maroccanus, Drosophila suzukii, Erionota thrax, Eriosomatinae, Eumetopina flavipes, European Corn Borer, Eurydema oleracea, Eurygaster integriceps, forest bug, Frankliniella occidentalis, Frankliniella triad, Galleria mellonella, garden dart, greenhouse whitefly, Gryllotalpa orientalis, Gryllus pennsylvanicus, gypsy moth, Helicoverpa armigera, Helicoverpa zea, Henosepilachna vigintioctopunctata, Hessian fly, Japanese beetle, Khapra beetle, Lampides boeticus, leaf miner, Lepidiota consobrina, Lepidosaphes ulmi, Leptoglossus zonatus, Leptopterna dolabrata, lesser wax moth, Leucoptera (moth), Leucoptera caffeina, light brown apple moth, Lissorhoptrus oryzophilus, long-tailed Skipper, Lygus, Maconellicoccus hirsutus, Macrodactylus subspinosus, Macrosiphum euphorbiae, maize weevil, Manduca sexta, Mayetiola hordei, mealybug, leek moth, Myzus persicae, Nezara viridula, olive fruit fly, Opomyzidae, Papilio demodocus, Paracoccus marginatus, Paratachardina pseudolobata, pea aphid, Pentatomoidea, Phthorimaea operculella, Phyllophaga (genus), Phylloxera, Phylloxeroidea, pink bollworm, Platynota idaeusalis, Plum curculio, Pseudococcus viburni, Pyralis farinalis, red imported fire ant, red locust, Rhagoletis cerasi, Rhagoletis indifferens, Rhagoletis mendax, Rhynchophorus ferrugineus, Rhyzopertha dominica, rice moth, Russian wheat aphid, San Jose scale, scale insect, Sciaridae, Scirtothrips dorsalis, Scutelleridae, serpentine leaf miner, silverleaf whitefly, small hive beetle, soybean aphid, Spodoptera cilium, Spodoptera litura, spotted cucumber beetle, squash vine borer, Stenotus binotatus, Sternorrhyncha, Strauzia longipennis, striped flea beetle, sunn pest, sweet potato bug, tarnished plant bug, Thrips palmi, Toxoptera citricida, Trioza erytreae, Tuta absoluta, varied carpet beetle, Virachola isocrates, waxworm, western corn rootworm, wheat weevil, winter moth and Xyleborus glabratus.

### Example 10—Preparation of Compositions Compromising Lipopeptides Using Submerged Co-Cultivation

In one embodiment, compositions comprising lipopeptides (e.g., surfactin) are produced using co-cultivation of Bacillus amyloliquefaciens and Myxococcus xanthus. When grown together, the species try to inhibit one another, thereby producing large amounts of lipopeptides with strong antibacterial properties.

Bacillus amyloliquefaciens inoculum is grown in a small-scale reactor for 24 to 48 hours. Myxococcus xanthus inoculum is grown in a 2 L working volume seed culture flask for 48 to 120 hours. A fermentation reactor is inoculated with the two inocula. The nutrient medium comprises:

| | |
|---|---|
| Glucose | 1 g/L to 5 g/L |
| Casein peptone | 1 g/L to 10 g/L |
| K₂HPO₄ | 0.01 g/L to 1.0 g/L |
| KH₂PO₄ | 0.01 g/L to 1.0 g/L |
| MgSO₄•7H₂O | 0.01 g/L to 1.0 g/L |
| NaCl | 0.01 g/L to 1.0 g/L |
| CaCO₃ | 0.5 g/L to 5 g/L |
| Ca(NO₃)₂ | 0.01 g/L to 1.0 g/L |
| Yeast extract | 0.01 g/L to 5 g/L |
| MnCl₂•4H₂O | 0.001 g/L to 0.5 g/L |
| Teknova trace | 0.5 ml/L to 5 ml/L |
| element | |

Fine grain particulate anchoring carrier is suspended in the nutrient medium. The carrier comprises cellulose (1.0 to 5.0 g/L) and/or corn flour (1.0 to 8.0 g/L).

The two species of bacteria produce lipopeptides into the liquid fermentation medium. The post-fermentation broth is then dried to remove excess water and inactivate the bacterial cells. This product, comprising nutrient medium, cells, and lipopeptides, is more beneficial for soil and plants than lipopeptides alone because the inactivated yeast cells are good sources of nutrients such as, for example, organic nitrogen, phosphorus and potassium.

### Example 11—Lipopeptides for Pest Control

Surfactin has the ability to reduce surface tension of water from 72 to 27 mN/m at a concentration as low as 0.005%. Surfactin has strong antibacterial (including antibiofilm), antiviral, and antimycoplasma activity, but less antifungal activity.

Iturin is a class of pore-forming lipopeptides having antifungal activity against a wide variety of pathogenic yeasts and fungi. Iturin can increase the microbial membrane cell permeability by the formation of ion-conducting pores. The antifungal activity is related to the interaction of the iturin lipopeptides with the cytoplasmic membrane of target cells and the K+ permeability of which is greatly increased.

Fengycins also display strong antifungal activity and inhibit the growth of a wide range of plant pathogens, particularly filamentous fungi.

Compositions comprising one or more of these lipopeptides can inhibit growth of, e.g., Botrytis cinerea, Sclerotinia sclerotiorum, Colletotrichum gloeosporioides, Phoma complanata, Fusarium spp., Aspergillus spp., Biopolaris sorokiniana, Xylella fastidiosa and Monilinia spp. (e.g., M. laxa and M. fructicola).

### Example 12—Biosurfactants With Microbial Cells

Advantageously, compositions according to the subject invention comprising microbial cell cultures are safe for application in the presence of plants, humans and animals. Inactivated microbial cells can contain high concentrations of protein, RNA, lipids, amino acids, vitamins, minerals and trace elements.

Preferably, the substrates used for producing microbial cultures according to the subject invention are all food-grade, safe products. Upon finishing the fermentation cycle, the resulting composition containing the produced biosurfactant, microbial cells, and residual broth and/or solid substrates can be dried to evaporate the excessive water excess and inactivate the cells. The resulting dried mass can be used as a final agricultural product and/or mixed with other dried microbial cultures.

In one exemplary embodiment, S. bombicola cells can be a source of nitrogen, phosphorus and potassium, which are valuable plant nutrients. Furthermore, the yeasts can be enriched with metals such as iron, copper and zinc during cultivation, thus providing a source of these nutrients for plants when applied to soil.

In one embodiment, a composition comprising S. bombicola cells, whether live or inactive, can increase the uptake of nutrients from soil by plant roots, leading to increases in root and shoot size. Additionally, in one embodiment, a composition comprising S. bombicola hydrolysate can help prevent bacterial and/or fungal diseases due to, for example, enhancement and/or activation of a plant's natural defensive mechanisms, in addition to the presence of antibacterial and/or antifungal biosurfactants and other metabolites in the culture.

### Example 13—Reducing Greenhouse Gases

There are three main greenhouse gases: carbon dioxide, methane and nitrous oxide. In certain embodiments, the methods and compositions of the subject invention help enhance agricultural practices in ways that reduce the emission of these and other polluting atmospheric gases.

In one embodiment, the compositions serve as replacements for chemical fertilizers, herbicides, pesticides, fumigants, fungicides, and growth stimulators, which may serve as precursor compounds for emission of atmospheric greenhouse gases.

In one embodiment, the compositions and methods of the subject invention methods reduce atmospheric carbon dioxide. Biosurfactants serve as growth enhancers, allowing for increased root and shoot size of plants. Thus, the healthier and more robust plants act as carbon sinks, fixing carbon during photosynthesis and storing excess carbon as biomass.

In one embodiment, the methods reduce methane emissions. Methane is produced by methanogenic archaea and bacteria in the digestive system of ruminant livestock. Compositions according to the subject invention, when applied to grazing pastures and/or livestock feed and then ingested by the animals, can reduce the amount of methanogenic organisms in the animals' digestive tracts, thereby reducing methane production.

In one embodiment, the methods reduce nitrous oxide emissions. About 60% of atmospheric nitrous oxide is produced by agricultural practices that utilize nitrate- and nitrite-based fertilizers, which are converted to nitrous oxide. Compositions according to the subject invention, when applied as replacement for mineral fertilizers, can thus reduce the amount of nitrous oxide produced by the agriculture industry.

### Example 14—Solid State “Matrix” Fermentation

A method of cultivating a microorganism and/or producing a microbial growth by-product can comprise: spreading a layer of a solid substrate mixed with water and, optionally, nutrients to enhance microbial growth, onto a tray to form a matrix; applying an inoculant of the microorganism onto the surface of the matrix; placing the inoculated tray into a fermentation reactor; passing air through the reactor to stabilize the temperature between 25-40° C.; and allowing the microorganism to propagate throughout the matrix.

In preferred embodiments, the matrix substrate according to the subject methods comprises foodstuffs. The foodstuffs can include, for example, rice, beans or legumes, lentils, quinoa, flaxseed, chia, corn, other grains, pasta, wheat bran, flours or meals (e.g., corn flour, nixtamilized corn flour, partially hydrolyzed corn meal), and/or other similar foodstuffs to provide surface area for the microbial culture to grow and/or feed on.

In one embodiment, the method of cultivation comprises preparing the trays, which can be, e.g., metal sheet pans or steam pans fitted for a standard proofing oven. In some embodiments, the “trays” can be any vessel or container capable of holding the substrate and culture, such as, for example, a flask, cup, bucket, plate, pan, tank, barrel, dish or column, made of, for example, plastic, metal or glass.

Preparation can comprise covering the inside surfaces of the trays with, for example, foil. Preparation can also comprise sterilizing the trays by, for example, autoclaving them.

Next, a matrix substrate is prepared by mixing a foodstuff item, water, and optionally, additional salts and/or nutrients to support microbial growth.

The mixture is then spread onto the trays and layered to form a matrix with a thickness of approximately 1 to 12 inches, preferably, 1 to 6 inches. The thickness of the matrix can vary depending on the volume of the tray or other container in which is it being prepared.

In preferred embodiments, the matrix substrate provides ample surface area on which microbes can grow, as well as enhanced access to oxygen supply. Thus, the substrate on which the microbes grow and propagate can also serve as the nutrient medium for the microbes.

The inoculated trays can then be placed inside a fermentation reactor in the form of a temperature-controlled space. Fermentation parameters can be adjusted based on the desired product to be produced (e.g., the desired microbial biosurfactant) and the microorganism being cultivated.

The temperature within the reactor depends upon the microorganism being cultivated, although in general, it is kept between about 25-40° C. using temperature-crontrolled air circulation. The circulating air can also provide continuous oxygenation to the culture. The air circulation can also help keep the DO at desired levels, for example, about 90% of ambient air.

The culture can be incubated for an amount of time that allows for the microorganism to reach a desired concentration, preferably from 1 day to 14 days, more preferably, from 2 days to 10 days.

In some embodiments, the microorganisms will consume either a portion of, or the entirety of, the matrix substrate throughout fermentation.

1-6. (canceled)

7. A method of enhancing soil health and/or plant health, the method comprising applying a composition comprising a live and/or inactivated Starmerella bombicola, Saccharomyces cerevisiae, Bacillus mojavensis, Burkholderia thailandensis, Pseudozyma aphidis, Bacillus amyloliquefaciens and/or Myxococcus xanthus microorganism, growth by-products thereof, optionally, a fermentation broth and/or solid-state substrate in which the microorganism was cultivated, and optionally, one or more sources of prebiotics, to the soil and/or plant.

8. The method of claim 7, wherein the one or more sources of prebiotics are kelp extract, fulvic acid, chitin, humate and/or humic acid.

9. The method of claim 7, wherein the growth by-products are biosurfactants.

10. The method of claim 9, wherein the biosurfactants produced by the S. bombicola, B. thailandensis, P. aphidis and S. cerevisiae are glycolipids.

11. The method of claim 10, wherein the glycolipids are rhamnolipids, mannosylerythritol lipids and/or sophorolipids.

12. The method of claim 9, wherein the biosurfactants produced by the B. mojavensis, B. amyloliquefaciens and M. xanthus are lipopeptides.

13. The method of claim 12, wherein the lipopeptides are fengycin, iturin and/or surfactin.

14. The method of claim 7, wherein one or more qualities of the soil are improved.

15. The method of claim 14, wherein the water retention capability of the soil is improved.

16. The method of claim 14, wherein the water drainage and/or dispersal capabilities of the soil are improved.

17. The method of claim 14, wherein the nutrient content of the soil is improved.

18. The method of claim 14, wherein pollutants in the soil are reduced and/or removed.

19. The method of claim 14, wherein salinity of the soil is reduced.

20. The method of claim 14, wherein the osmotic pressure in the soil is reduced.

21. The method of claim 7, used to control a pest.

22. The method of claim 7, used to activate a plant's defensive mechanisms.

23. The method of claim 7, used to stimulate the growth of a plant.

24. The method of claim 7, wherein GHG emissions are reduced.

25. The method of claim 7, further comprising characterizing the soil type prior to applying the composition to the soil.
